(19) United States
(12) Patent Application Publication
(10) Pub. No.: US 2023/0201360 A1
(43) Pub. Date: Jun. 29, 2023

(54) Composition and Kit for colonization in gut of external probiotics through bioorthogonal click chemistry

(71) Applicant: RESEARCH & BUSINESS FOUNDATION SUNGKYUNKWAN UNIVERSITY, Suwon-si (KR)

(72) Inventors: Jae Hyung PARK, Suwon-si (KR); Wooram UM, Hwaseong-si (KR); Hyeyeon JOO, Suwon-si (KR)

(73) Assignee: RESEARCH & BUSINESS FOUNDATION SUNGKYUNKWAN UNIVERSITY, Suwon-si (KR)

(21) Appl. No.: 18/077,484

(22) Filed: Dec. 8, 2022

(30) Foreign Application Priority Data
Dec. 10, 2021 (KR) 10-2021-0176780
Nov. 23, 2022 (KR) 10-2022-0158706

(51) Int. Cl. A61K 47/54; A61K 35/745; A61K 35/747; A61P 1/14

(52) U.S. Cl. CPC A61K 47/555; A61K 35/745; A61K 35/747; A61P 1/14

(57) ABSTRACT

The present disclosure relates to a composition and a kit for colonization of external probiotics in the gut through bioorthogonal click chemistry. The composition or kit for colonization of external probiotics in the gut according to the present disclosure has the effects of overcoming colonization resistance, which is the limitation of existing external probiotics caused by physical discharge and indigenous bacteria, by linking surface-modified external probiotics to the intestinal mucosa into which a bioorthogonal functional group has been introduced, and beneficially changing the intestinal environment by long-term colonization of various external probiotics, such as lactic acid bacteria, bifidobacteria, and yeast. Accordingly, the present disclosure is expected to be effectively used for the development of various medicines and functional foods using probiotics.

### TECHNICAL FIELD

The present disclosure relates to a composition and a kit for colonization of external probiotics in the gut through bioorthogonal click chemistry.

This application claims priority based on Korean Patent Application No. 10-2021-0176780, filed on Dec. 10, 2021, and Korean Patent Application No. 10-2022-0158706, filed on Nov. 23, 2022, all contents of which that are disclosed in the specification and drawings of the application are incorporated in this application.

### BACKGROUND ART OF INVENTION

The term microbiome refers to the population of all microorganisms present in a specific environment. In the case of the human body, most of the microbiome is distributed in the intestine, and about 100 trillion microorganisms constituting the same are closely associated with activity control of various cells in the body through various metabolites. It has been elucidated that changes in the population of gut microbiome are deeply associated with health problems that do not seem to have much to do with microbes, such as obesity, diabetes, cancer, allergies, and various neurological diseases. It is known that not only inflammatory bowel diseases such as Crohn's disease and ulcerative colitis, but also various intestinal diseases such as diarrhea, abdominal pain, and colon cancer are associated with the imbalance of intestinal microflora, and it has been reported that depending on the environment of the gut microbiome, such changes can have an effect on the occurrence of allergic diseases (e.g., atopic dermatitis), neurological diseases (e.g., depression and dementia), and metabolic diseases (e.g., obesity and diabetes) by way of regulation of human gene expression or secretion of substances involved in inflammation. Therefore, among the intestinal microbiome, there has been a significantly growing interest in probiotics that produce metabolites beneficial to the body.

Probiotics is a generic term referring to living microorganisms that produce various beneficial effects on host animals when ingested, and mainly include lactic acid bacteria. Lactic acid bacteria (LAB) include Lactobacillus sp., Lactococcus sp., Enterococcus sp., Streptococcus sp., and Pediococcus sp., Leuconostoc sp., Sporolactobacilus sp., Bifidobacterium sp., etc. After entering the intestine, these lactic acid bacteria not only adhere to intestinal epithelial cells to prevent the colonization of harmful microorganisms and secrete antibacterial substances to inhibit the growth of harmful microorganisms and improve diarrhea and constipation, but also have the effects of enhancing immune activity, anti-cancer activity, etc. Since lactic acid bacteria enter the intestine and secrete substances through their own metabolic activities, many studies using lactic acid bacteria are underway due to their various benefits to the host. Lactic acid bacteria normally exist in the intestines of most animals, maintain the balance of intestinal microorganisms, and have beneficial effects on the health of the hosts.

As a strategy for increasing the intestinal ratio of probiotics, the method of ingesting external probiotics is most widely used. However, in the case of the intestinal microbiome, indigenous bacteria form homogeneous colonies and have colonization resistance that inhibits the colonization of new bacteria introduced from the outside. Therefore, most external probiotics do not colonize the intestinal environment but are excreted or eliminated within 2 to 5 days after ingestion, and thus the effect is merely temporary and has a limitation in that it does not affect long-term changes in the intestinal environment.

In this regard, the present inventors have attempted to colonize the intestine for a long period of time by linking the external probiotics to the intestinal mucosa by way of bioorthogonal click chemistry.

### DISCLOSURE OF INVENTION

Technical Problem

The present inventors have conducted studies to solve the problem in that conventional external probiotics, when ingested, cannot colonize the intestinal environment. As a result, they have developed a method for long-term intestinal colonization of external probiotics through selective introduction of external probiotics to the intestinal mucosa by administering a precursor that imparts a bioorthogonal functional group to the intestinal mucosa to form an intestinal mucosa in which a bioorthogonal functional group is introduced, and by administering external probiotics modified to bind to the bioorthogonal functional group through click chemistry, thereby completing the present disclosure based on the discovery.

Accordingly, an object of the present disclosure is to provide a composition and a kit for colonization of external probiotics in the gut.

However, the technical problems to be achieved by the present disclosure are not limited to the problems mentioned above, and other problems not mentioned will be clearly understood by those skilled in the art to which the present disclosure belongs from the description below.

[Technical Solution]

In order to achieve the objects described above, the present disclosure provides a composition for colonization of external probiotics in the gut, which comprises as active ingredients:

a first composition comprising a precursor that imparts a bioorthogonal functional group to the intestinal mucosa; and

a second composition comprising probiotics modified with a functional group that selectively binds to the bioorthogonal functional group.

Additionally, the present disclosure provides a kit for colonization of external probiotics in the gut, which comprises:

a first container comprising the first composition;

a second container comprising a second composition; and

an instruction manual.

In an embodiment of the present disclosure, the first composition, when administered to a subject, may form a functional group on the intestinal mucosa by a precursor that imparts a bioorthogonal functional group to thereby change the composition of the intestinal mucosa, and

the second composition, when administered to a subject, may enable selective colonization in the intestinal mucosa of the probiotics modified with a functional group that selectively binds to a bioorthogonal functional group through a specific chemical reaction, but the first and compositions are not limited thereto.

In another embodiment of the present disclosure, the specific chemical reaction may be a bioorthogonal click chemistry reaction, but is not limited thereto.

In still another embodiment of the present disclosure, the bioorthogonal functional group may be an azide group (—N₃), but is not limited thereto.

In still another embodiment of the present disclosure, the precursor that imparts a bioorthogonal functional group may be one or more selected from the group consisting of tetraacetylated N-azidoacetyl-D-galactosamine (GalNAz), tetraacetylated N-azidoacetyl-D-mannos amine (ManNAz), tetraacetylated N-azidoacetyl-D-glucosamine, tetraacetylated N-azidoacetyl-D-galactose, tetraacetylated N-azidoacetyl-D-mannose, and tetraacetylated N-azidoacetyl-D-glucose, but is not limited thereto.

In still another embodiment of the present disclosure, the functional group that selectively binds to a bioorthogonal functional group may be one or more selected from the group consisting of dibenzocyclooctyne (DBCO), cyclooctyne, 2,2-difluoro-1,3-cyclooctanedione, difluorinated cyclooctyne, bicyclo[6.1.0]non-2-yne or a derivative thereof, 4-dibenzocyclooctinol,

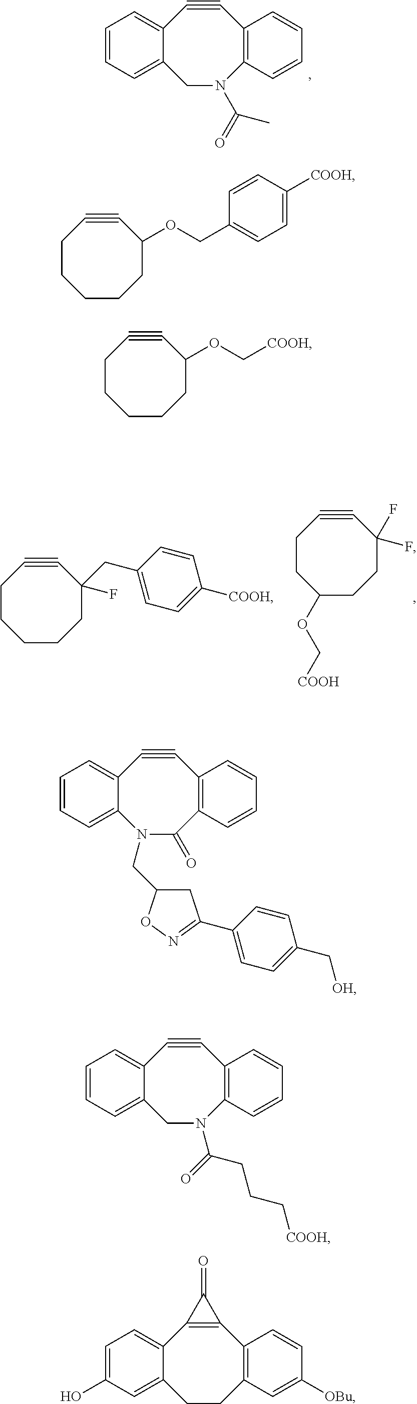

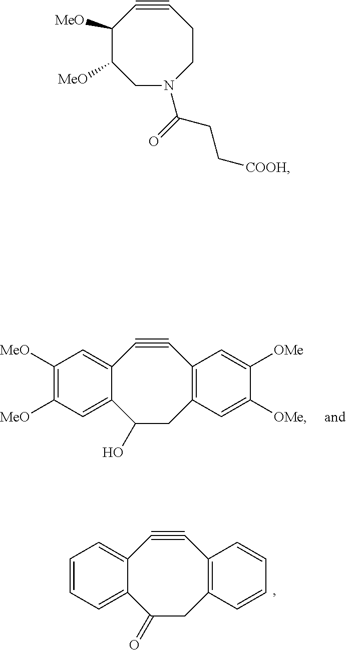 [SMILES: CO[C@H]1C#CCCN(C(=O)CCC(=O)O)C[C@@H]1OC ; COc1cc2c(cc1OC)CC(O)c1cc(OC)c(OC)cc1C#C2 ; O=C1Cc2ccccc2C#Cc2ccccc21]

but is not limited thereto.

In still another embodiment of the present disclosure, the composition may introduce external probiotics into the intestine and improves the intestinal environment while maintaining the structure and function of the external probiotics, but is not limited thereto.

In still another embodiment of the present disclosure, the molar concentration of the functional group that selectively binds to the bioorthogonal functional group of the second composition may be 3-12 fold that of the precursor imparting the bioorthogonal functional group of the first composition relative to the composition, but is not limited thereto.

In still another embodiment of the present disclosure, the formulation of the first composition or second composition may be at least one selected from the group consisting of powders, granules, sustained-release granules, enteric-coated granules, solutions, emulsions, suspensions, spirits, troches, perfumes, tablets, sustained-release tablets, enteric-coated tablets, sublingual tablets, hard capsules, soft capsules, sustained-release capsules, enteric-coated capsules, pills, tinctures, soft extracts, dried extracts, fluid extracts, injections, and perfusion solutions, but is not limited thereto.

In still another embodiment of the present disclosure, the instruction manual may describe that the first composition is first administered to a subject in need thereof, and 12 to 48 hours thereafter, the second composition is first administered, but is not limited thereto.

In still another embodiment of the present disclosure, the instruction manual may further describe that after the first administration of the first composition, it is possible to administer the first composition and the second composition in combination or to administer the second composition after the administration of the first composition, but is not limited thereto.

In still another embodiment of the present disclosure, the instruction manual may further describe that it is possible to administer the second composition 2 to 3 times repeatedly per one administration of the first composition, but is not limited thereto.

In still another embodiment, the present disclosure may provide a method for colonization of external probiotics in the gut, which comprises administering a first composition comprising a precursor that imparts a bioorthogonal functional group to the intestinal mucosa to a subject in need thereof; and administering a second composition comprising probiotics modified with a functional group that selectively binds to the bioorthogonal functional group to the subject.

Additionally, the present disclosure provides a use of a composition for colonization of external probiotics in the gut, in which the composition comprises, as active ingredients, a first composition comprising a precursor that imparts a bioorthogonal functional group to the intestinal mucosa; and a second composition comprising probiotics modified with a functional group that selectively binds to the bioorthogonal functional group.

Additionally, the present disclosure provides a use of a first composition comprising a precursor that imparts a bioorthogonal functional group to the intestinal mucosa and a second composition comprising probiotics modified with a functional group that selectively binds to the bioorthogonal functional group for the preparation of an agent for colonization of external probiotics in the gut.

Additionally, the present disclosure provides a use of a composition for the preparation of an agent for colonization of external probiotics in the gut, in which the composition comprises, as active ingredients, a first composition comprising a precursor that imparts a bioorthogonal functional group to the intestinal mucosa; and a second composition comprising probiotics modified with a functional group that selectively binds to the bioorthogonal functional group.

### Effects of Invention

The composition or kit for colonization of external probiotics in the gut according to the present disclosure has the effects of overcoming colonization resistance, which is a limitation of existing external probiotics caused by physical discharge and indigenous bacteria, by linking surface-modified external probiotics to the intestinal mucosa into which a bioorthogonal functional group has been introduced, and beneficially changing the intestinal environment by long-term colonization of various external probiotics, such as lactic acid bacteria, bifidobacteria, and yeast.

### DESCRIPTION OF DRAWINGS

FIG. 1 shows schematic diagrams illustrating the introduction of external probiotics into the intestinal mucosa using bioorthogonal click chemistry according to an embodiment of the present disclosure.

FIG. 2A shows graphs confirming, by flow cytometry, whether or not DBCO is introduced into probiotics by DBCO-NHS treatment according to an embodiment of the present disclosure.

FIG. 2B shows images confirming, by fluorescence images, whether or not DBCO is introduced (DBCO-Bac.) in probiotics by DBCO-NHS treatment according to an embodiment of the present disclosure.

FIG. 3A shows images confirming, by a transmission electron microscope, the structural characteristics of probiotics before and after the introduction of DBCO according to an embodiment of the present disclosure.

FIG. 3B shows a graph confirming the viability of DBCO-Bac. by DBCO concentration according to an embodiment of the present disclosure.

FIG. 4A shows images confirming, by fluorescence images, the expression of an azide group in goblet cell-like cells by GalNAz or ManNAz treatment according to an embodiment of the present disclosure.

FIG. 4B shows results confirming, by Western blot, the expression of an azide group in goblet cell-like cells by concentration of GalNAz or ManNAz according to an embodiment of the present disclosure.

FIG. 5A shows graphs and images confirming the binding of GalNAz- or ManNAz-treated goblet cell-like cells and DBCO-Bac. according to an embodiment of the present disclosure by fluorescence images (left figure) and flow cytometry (right figure).

FIG. 5B shows images confirming, by fluorescence images, DBCO-Bac bound with GalNAz- or ManNAz-treated goblet cell-like cells according to an embodiment of the present disclosure.

FIG. 6 shows an image confirming, by fluorescence images, DBCO-Bac bound with GalNAz- or ManNAz-treated goblet cell-like cells in a three-dimensional intestinal mucosal simulation model according to an embodiment of the present disclosure.

FIG. 7A shows graphs and images confirming, by fluorescence images, the expression level of an azide group over time in the duodenum, jejunum, ileum, and colon of the small intestine after oral administration of GalNAz or ManNAz to mice according to an embodiment of the present disclosure.

FIG. 7B shows graphs and images confirming the amount of fluorescence of Bac. remaining in the intestinal mucosa according to post-treatment time after oral administration of GalNAz or ManNAz to mice and DBCO-Bac. treatment according to an embodiment of the present disclosure.

FIG. 7C shows images confirming the amount of fluorescence of Bac. remaining in the intestinal mucosa after oral administration of GalNAz or ManNAz to mice, and simultaneous treatment with DBCO-modified Bifidobacterium longum (orange fluorescence) and DBCO-modified Lactobacillus acidophilus (green fluorescence) according to an embodiment of the present disclosure.

### DETAILED DESCRIPTION OF INVENTION

In an experimental embodiment of the present disclosure, it was confirmed that when DBCO, which can react with an azide group in vivo without a catalyst, was introduced into the surface of probiotics by way of bioorthogonal click chemistry, the DBCO introduced was selectively distributed on the surface of probiotics (see Experimental Example 1).

In another experimental embodiment of the present disclosure, it was confirmed that the introduction of DBCO did not affect the structural characteristics of probiotics, and it was also confirmed that the introduction of DBCO did not affect the viability of probiotics. Therefore, it was found that the present disclosure can increase intestinal colonization of probiotics without functional loss of the same (see Experimental Example 2).

In still another experimental embodiment of the present disclosure, it was confirmed that an azide group, a functional group for bioorthogonal click chemistry, was produced in a concentration-dependent manner when GalNAz or ManNAz was treated on goblet cell-like cells (see Experimental Example 3).

In still another experimental embodiment of the present disclosure, it was confirmed that when DBCO-Bac was treated on goblet cell-like cells into which an azide group is introduced, the binding ability to DBCO-Bac. was significantly increased compared to the control group into which an azide group is not introduced. Therefore, it was confirmed that the present disclosure can selectively introduce specific external probiotics without affecting colonization resistance for inhibition of pathogens in vivo (see Experimental Example 4).

In still another experimental embodiment of the present disclosure, as a result of evaluating the binding ability of DBCO-Bac. to the intestinal mucosa in a three-dimensional environment using a three-dimensional intestinal mucosal simulation model, it was confirmed that when treated with GalNAz or ManNAz, the binding ability to DBCO-Bac. was significantly improved compared to the control group, and in particular, it was confirmed that not only the binding ability of the surface but also the internal binding ability increased in the mucosal structure (see Experimental Example 5).

In still another experimental embodiment of the present disclosure, as a result of confirming the expression level of an azide group over time in the duodenum, jejunum, ileum, and colon of the small intestine after oral administration of GalNAz or ManNAz to mice, it was confirmed that the expression level of an azide group was shown to be high in the intestinal mucosa about 24 hours after oral administration of GalNAz or ManNAz in each region of the intestine (see Experimental Example 6).

In still another experimental embodiment of the present disclosure, as a result of confirming the fluorescence amount of Bac. remaining in the intestinal mucosa according to the time after oral administration of GalNAz or ManNAz and DBCO-Bac. treatment to mice, it was confirmed that when treated with DBCO-Bac., both groups administered with GalNAz and ManNAz showed a higher amount of fluorescence remaining in the intestinal mucosa and a longer duration of fluorescence compared to the group administered with physiological saline, thus confirming that a large amount of Bac. can be maintained in the intestinal mucosa for a long time through the binding of DBCO to an azide group introduced into the intestinal mucosa caused by GalNAz and ManNAz (see Experimental Example 7).

In still another experimental embodiment of the present disclosure, as a result of confirming the fluorescence amount of Bac. remaining in the intestinal mucosa after oral administration of GalNAz or ManNAz and simultaneous treatment with DBCO-modified Bifidobacterium longum (orange fluorescence) and DBCO-modified Lactobacillus acidophilus (green fluorescence), it was confirmed that the amounts of Bifidobacterium longum and Lactobacillus acidophilus remaining in the intestinal mucosa were higher in the groups administered with GalNAz and ManNAz compared to the control group, thus confirming that external probiotics modified with DBCO can colonize the intestinal mucosa (see Experimental Example 8).

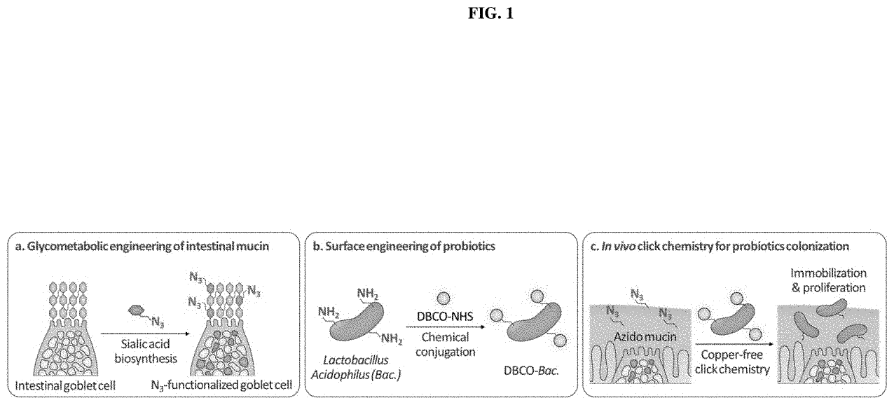

A schematic diagram regarding the process of introducing external probiotics into the intestinal mucosa by bioorthogonal click chemistry according to the present disclosure is shown in FIG. 1.

In this regard, the present disclosure provides a composition for colonization of external probiotics in the gut, which comprises as active ingredients: a first composition comprising a precursor that imparts a bioorthogonal functional group to the intestinal mucous membrane; and a second composition comprising probiotics modified with a functional group that selectively binds to the bioorthogonal functional group.

In the present disclosure, the first composition, when administered to a subject, forms a functional group on the intestinal mucous membrane by a precursor that imparts a bioorthogonal functional group to thereby change the composition of the intestinal mucous membrane, and the second composition, when administered to a subject, enables selective colonization in the intestinal mucous membrane of the probiotics modified with a functional group that selectively binds to a bioorthogonal functional group through a specific chemical reaction, but the first and second compositions are not limited thereto.

In the present disclosure, the specific chemical reaction may be a bioorthogonal click chemistry reaction, but is not limited thereto.

In the present disclosure, the term “colonization” refers to colonization of bacteria on the skin or mucous membranes without any specific symptoms of infection.

In the present disclosure, the term “bioorthogonal click chemistry” refers to a chemical reaction that can occur within a living system without interfering with natural biochemical processes, and certain bioorthogonal compounds, while not reacting with biomolecules in vivo, react only with specific non-natural molecules injected from the outside to thereby form a bond as if two different functional groups meet and “click”.

In the composition for colonization of external probiotics in the gut, the precursor that imparts the bioorthogonal functional group may be one or more selected from the group consisting of tetraacetylated N-azidoacetyl-D-galactosamine (GalNAz), tetraacetylated N-azidoacetyl-D-mannosamine (ManNAz), tetraacetylated N-azidoacetyl-D-glucosamine, tetraacetylated N-azidoacetyl-D-galactose, tetraacetylated N-azidoacetyl-D-mannose, and tetraacetylated N-azidoacetyl-D-glucose, and according to an embodiment of the present disclosure, may be at least one selected from the group consisting of tetraacetylated N-azidoacetyl-D-galactosamine represented by Formula 1 below and tetraacetylated N-azidoacetyl-D-mannosamine represented by Formula 2 below, but are not limited thereto. The precursor can impart a bioorthogonal functional group to intestinal mucosa by metabolic engineering of intestinal goblet cells. The metabolic engineering refers to introduction of a modified sugar chain into a cell using native metabolism.

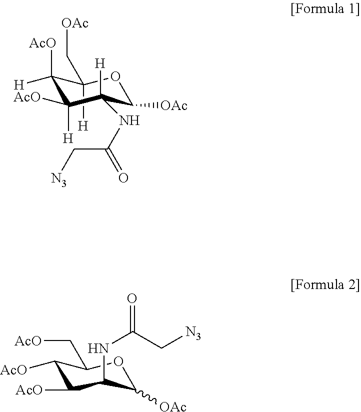

In the present disclosure, the bioorthogonal functional group being imparted to the intestinal mucosa refers to a functional group that causes a bioorthogonal click chemistry reaction, for example an azide group (-N₃), but is not limited thereto.

In the composition for colonization of external probiotics in the gut of the present disclosure, the functional group that selectively binds to a bioorthogonal functional group may be one or more selected from the group consisting of dibenzocyclooctyne (DBCO), cyclooctyne, 2,2-difluoro-1,3-cyclooctanedione, difluorinated cyclooctyne, bicyclo[6.1.0]non-2-yne or a derivative thereof, 4-dibenzocyclooctinol,

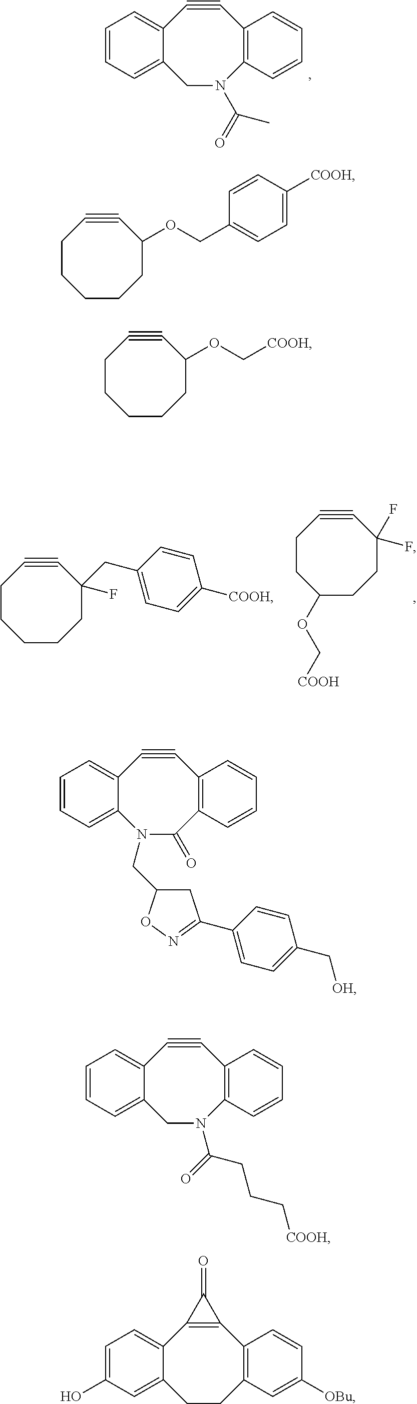

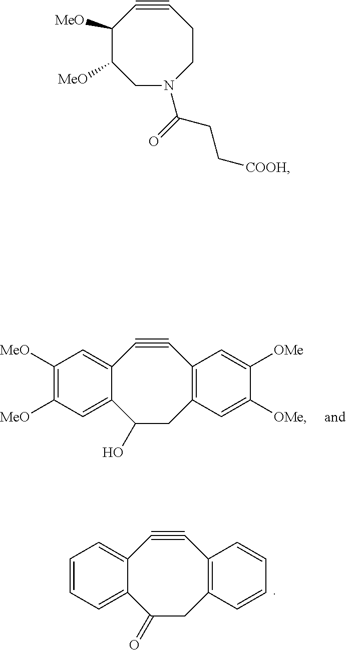 [SMILES: CO[C@H]1C#CCCN(C(=O)CCC(=O)O)C[C@@H]1OC ; COc1cc2c(cc1OC)CC(O)c1cc(OC)c(OC)cc1C#C2 ; O=C1Cc2ccccc2C#Cc2ccccc21]

According to an embodiment of the present disclosure, the functional group that selectively binds to a bioorthogonal functional group may be one or more selected from the group consisting of dibenzocyclooctyne and cyclooctyne, and dibenzocyclooctyne and cyclooctyne may be represented by Formula 3 and Formula 4 below, respectively, but are not limited thereto.

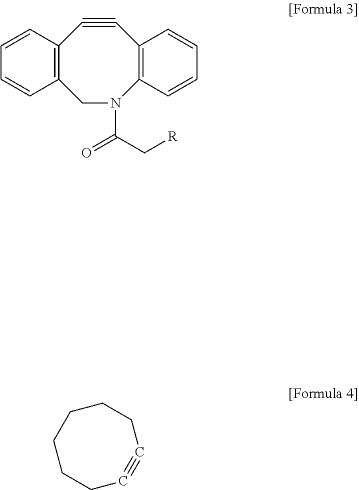

In the present disclosure, the term “probiotics” is a general term for beneficial bacteria that enter the body and provide a good effect on health. Most probiotics known to date are lactic acid bacteria, including some Bacillus sp. In the present disclosure, the term “external probiotics” refers to probiotics administered into the living body from the outside, in addition to probiotics present in vivo.

The probiotics may have an NH₂ functional group on the surface, and, for example, Lactobacillus sp. which includes Lactobacillus acidophilus, Lactobacillus casei, Lactobacillus gasseri, Lactobacillus delbrueckii ssp. bulgaricus, Lactobacillus helveticus, Lactobacillus fermentum, Lactobacillus paracasei, Lactobacillus plantarum, Lactobacillus reuteri, Lactobacillus rhamnosus, or Lactobacillus salivarius; Lactococcus sp. which includes Lactococcus lactis; Enterococcus sp. which includes Enterococcus faecium or Enterococcus faecalis; and Streptococcus sp. which includes Streptococcus thermophilus; or Bifidobacterium sp. which includes Bifidobacterium bifidum, Bifidobacterium breve, Bifidobacterium longum, or Bifidobacterium animalis ssp. Lactis, but the types of probiotics are not limited thereto.

In the present disclosure, as the probiotics, two or more types of bacterial groups may be administered, and there is no limitation in the order of administration of these bacteria, and simultaneous administration is also possible.

In the present disclosure, the composition for colonization of external probiotics in the gut may improve the intestinal environment by introducing external probiotics into the intestine while maintaining the structures and functions of the external probiotics, but is not limited thereto.

In the present disclosure, the term “maintenance” refers to a state in which the structure and functions of external probiotics are preserved or continue to exist.

In the present disclosure, the term “improvement of intestinal environment” refers to a beneficial change in the intestinal environment, and specifically means that probiotics, which have reached the intestine and are able to grow in the intestinal mucosa, produce lactic acid and makes the intestinal environment acidic; as a result, the number of harmful bacteria that cannot survive in an acidic environment are reduced, whereas the beneficial bacteria that grow well in an acidic environment proliferate more, thereby making the intestinal environment healthy.

In the present disclosure, with regard to the composition for colonization of external probiotics in the gut, the molar concentration of the functional group that selectively binds to a bioorthogonal functional group of the second composition may be contained 3- to 12-fold, 3- to 11-fold, 3- to 10-fold, 4- to 12-fold, 4- to 11-fold, 4- to 10-fold, 5- to 12-fold, 5- to 11-fold, 5- to 10-fold, 6- to 12-fold, 6- to 11-fold, 6- to 10-fold, 7- to 12-fold, 7- to 11-fold, 7- to 10-fold, 8- to 12-fold, 8- to 11-fold, 8- to 10-fold, 9- to 12-fold, 9- to 11-fold, 9- to 10-fold, 10- to 12-fold, 10- to 11-fold, or 10-fold, compared to the molar concentration ratio of the precursor that imparts a bioorthogonal functional group of the first composition, but is not limited thereto.

In the present disclosure, the molar concentration of a functional group that selectively binds to a bioorthogonal functional group of the second composition per 10¹¹ of probiotics may be 300 μM to 1,500 μM, 500 μM to 1,500 μM, 700 μM to 1,500 μM, 900 μM to 1,500 μM, 1,000 μM to 1,500 μM, 300 μM to 1,200 μM, 500 μM to 1,200 μM, 700 μM to 1,200 μM, 900 μM to 1,200 μM, 1,000 μM to 1,200 μM, 300 μM to 1,000 μM, 500 μM to 1,000 μM, 700 μM to 1,000 μM, 900 μM to 1,000 μM, or 1000 μM, but is not limited thereto.

In the present disclosure, the composition for colonization of external probiotics in the gut may be provided in the form of a pharmaceutical composition or food composition, and the food composition may be a health functional food composition, but is not limited thereto.

In the present disclosure, when the composition for colonization of external probiotics in the gut is provided in the form of a pharmaceutical composition, it may be used for prevention or treatment of various kinds of intestinal diseases (e.g., inflammatory bowel disease including Crohn's disease, ulcerative colitis, intestinal Behcet's disease, infectious enteritis, ischemic enteritis, or radiation enteritis, and irritable metabolic syndrome, etc.), or diabetes, hypertension, autoimmune disease, Alzheimer's disease, cancer, etc. which are affected by the composition of the gut microbiome.

The pharmaceutical composition according to the present invention may further include a suitable carrier, excipient, and diluent which are commonly used in the preparation of pharmaceutical compositions. The excipient may be, for example, one or more selected from the group consisting of a diluent, a binder, a disintegrant, a lubricant, an adsorbent, a humectant, a film-coating material, and a controlled release additive.

As the carrier, the excipient, and the diluent that may be included in the pharmaceutical composition according to the present invention, lactose, dextrose, sucrose, oligosaccharides, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, and mineral oil may be used.

For formulation, commonly used diluents or excipients such as fillers, thickeners, binders, wetting agents, disintegrants, and surfactants are used.

As additives of tablets, powders, granules, capsules, pills, and troches according to the present invention, excipients such as corn starch, potato starch, wheat starch, lactose, white sugar, glucose, fructose, D-mannitol, precipitated calcium carbonate, synthetic aluminum silicate, dibasic calcium phosphate, calcium sulfate, sodium chloride, sodium hydrogen carbonate, purified lanolin, microcrystalline cellulose, dextrin, sodium alginate, methyl cellulose, sodium carboxymethylcellulose, kaolin, urea, colloidal silica gel, hydroxypropyl starch, hydroxypropyl methylcellulose (HPMC), HPMC 1928, HPMC 2208, HPMC 2906, HPMC 2910, propylene glycol, casein, calcium lactate, and Primojel®; and binders such as gelatin, Arabic gum, ethanol, agar powder, cellulose acetate phthalate, carboxymethylcellulose, calcium carboxymethylcellulose, glucose, purified water, sodium caseinate, glycerin, stearic acid, sodium carboxymethylcellulose, sodium methylcellulose, methylcellulose, microcrystalline cellulose, dextrin, hydroxycellulose, hydroxypropyl starch, hydroxymethylcellulose, purified shellac, starch, hydroxypropyl cellulose, hydroxypropyl methylcellulose, polyvinyl alcohol, and polyvinylpyrrolidone may be used, and disintegrants such as hydroxypropyl methylcellulose, corn starch, agar powder, methylcellulose, bentonite, hydroxypropyl starch, sodium carboxymethylcellulose, sodium alginate, calcium carboxymethylcellulose, calcium citrate, sodium lauryl sulfate, silicic anhydride, 1-hydroxypropylcellulose, dextran, ion-exchange resin, polyvinyl acetate, formaldehyde-treated casein and gelatin, alginic acid, amylose, guar gum, sodium bicarbonate, polyvinylpyrrolidone, calcium phosphate, gelled starch, Arabic gum, amylopectin, pectin, sodium polyphosphate, ethyl cellulose, white sugar, magnesium aluminum silicate, a di-sorbitol solution, and light anhydrous silicic acid; and lubricants such as calcium stearate, magnesium stearate, stearic acid, hydrogenated vegetable oil, talc, lycopodium powder, kaolin, Vaseline, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydride, higher fatty acids, higher alcohols, silicone oil, paraffin oil, polyethylene glycol fatty acid ether, starch, sodium chloride, sodium acetate, sodium oleate, dl-leucine, and light anhydrous silicic acid may be used.

As additives of liquids according to the present invention, water, dilute hydrochloric acid, dilute sulfuric acid, sodium citrate, monostearic acid sucrose, polyoxyethylene sorbitol fatty acid esters (twin esters), polyoxyethylene monoalkyl ethers, lanolin ethers, lanolin esters, acetic acid, hydrochloric acid, ammonia water, ammonium carbonate, potassium hydroxide, sodium hydroxide, prolamine, polyvinylpyrrolidone, ethylcellulose, and sodium carboxymethylcellulose may be used.

In syrups according to the present invention, a white sugar solution, other sugars or sweeteners, and the like may be used, and as necessary, a fragrance, a colorant, a preservative, a stabilizer, a suspending agent, an emulsifier, a viscous agent, or the like may be used.

In emulsions according to the present invention, purified water may be used, and as necessary, an emulsifier, a preservative, a stabilizer, a fragrance, or the like may be used.

In suspensions according to the present invention, suspending agents such as acacia, tragacanth, methylcellulose, carboxymethylcellulose, sodium carboxymethylcellulose, microcrystalline cellulose, sodium alginate, hydroxypropyl methylcellulose (HPMC), HPMC 1828, HPMC 2906, HPMC 2910, and the like may be used, and as necessary, a surfactant, a preservative, a stabilizer, a colorant, and a fragrance may be used.

Injections according to the present invention may include: solvents such as distilled water for injection, a 0.9% sodium chloride solution, Ringer's solution, a dextrose solution, a dextrose+sodium chloride solution, PEG, lactated Ringer's solution, ethanol, propylene glycol, non-volatile oil-sesame oil, cottonseed oil, peanut oil, soybean oil, corn oil, ethyl oleate, isopropyl myristate, and benzene benzoate; cosolvents such as sodium benzoate, sodium salicylate, sodium acetate, urea, urethane, monoethylacetamide, butazolidine, propylene glycol, the Tween series, amide nicotinate, hexamine, and dimethylacetamide; buffers such as weak acids and salts thereof (acetic acid and sodium acetate), weak bases and salts thereof (ammonia and ammonium acetate), organic compounds, proteins, albumin, peptone, and gums; isotonic agents such as sodium chloride; stabilizers such as sodium bisulfite (NaHSO₃) carbon dioxide gas, sodium metabisulfite (Na₂S₂O₅), sodium sulfite (Na₂SO₃), nitrogen gas (N₂), and ethylenediamine tetraacetic acid; sulfating agents such as 0.1% sodium bisulfide, sodium formaldehyde sulfoxylate, thiourea, disodium ethylenediaminetetraacetate, and acetone sodium bisulfite; a pain relief agent such as benzyl alcohol, chlorobutanol, procaine hydrochloride, glucose, and calcium gluconate; and suspending agents such as sodium CMC, sodium alginate, Tween 80, and aluminum monostearate.

In suppositories according to the present invention, bases such as cacao butter, lanolin, Witepsol, polyethylene glycol, glycerogelatin, methylcellulose, carboxymethylcellulose, a mixture of stearic acid and oleic acid, Subanal, cottonseed oil, peanut oil, palm oil, cacao butter +cholesterol, lecithin, lanette wax, glycerol monostearate, Tween or span, imhausen, monolan(propylene glycol monostearate), glycerin, Adeps solidus, buytyrum Tego-G, cebes Pharma 16, hexalide base 95, cotomar, Hydrokote SP, 5-70-XXA, S-70-XX75(S-70-XX95), Hydrokote 25, Hydrokote 711, idropostal, massa estrarium (A, AS, B, C, D, E, I, T), masa-MF, masupol, masupol-15, neosuppostal-N, paramount-B, supposiro OSI, OSIX, A, B, C, D, H, L, suppository base IV types AB, B, A, BC, BBG, E, BGF, C, D, 299, suppostal N, Es, Wecoby W, R, S, M, Fs, and tegester triglyceride matter (TG-95, MA, 57) may be used.

Solid preparations for oral administration include tablets, pills, powders, granules, capsules, and the like, and such solid preparations are formulated by mixing the composition with at least one excipient, e.g., starch, calcium carbonate, sucrose, lactose, gelatin, and the like. In addition to simple excipients, lubricants such as magnesium stearate and talc are also used.

Examples of liquid preparations for oral administration include suspensions, liquids for internal use, emulsions, syrups, and the like, and these liquid preparations may include, in addition to simple commonly used diluents, such as water and liquid paraffin, various types of excipients, for example, a wetting agent, a sweetener, a fragrance, a preservative, and the like. Preparations for parenteral administration include an aqueous sterile solution, a non-aqueous solvent, a suspension, an emulsion, a freeze-dried preparation, and a suppository. Non-limiting examples of the non-aqueous solvent and the suspension include propylene glycol, polyethylene glycol, a vegetable oil such as olive oil, and an injectable ester such as ethyl oleate.

In the present disclosure, when the composition for colonization of external probiotics in the gut is provided in the form of a food composition, it can be used for preventing or improving various intestinal diseases including inflammatory bowel disease, or improving the intestinal environment. The composition for colonization of external probiotics in the gut according to the present invention may be used by adding the composition for colonization of external probiotics in the gut as is to food or may be used together with other foods or food ingredients, but may be appropriately used according to a typical method. The mixed amount of the active ingredient may be suitably determined depending on the purpose of use thereof (for prevention or alleviation). In general, when a food or beverage is prepared, the composition for colonization of external probiotics in the gut of the present invention is added in an amount of 15 wt% or less, preferably 10 wt% or less based on the raw materials. However, for long-term intake for the purpose of health and hygiene or for the purpose of health control, the amount may be less than the above-mentioned range, and the vesicles have no problem in terms of stability, so the active ingredient may be used in an amount more than the above- mentioned range.

The type of food is not particularly limited. Examples of food to which the material may be added include meats, sausage, bread, chocolate, candies, snacks, confectioneries, pizza, instant noodles, other noodles, gums, dairy products including ice creams, various soups, beverages, tea, drinks, alcoholic beverages, vitamin complexes, and the like, and include all health functional foods in a typical sense.

The health beverage composition according to the present invention may contain various flavors or natural carbohydrates, and the like as additional ingredients as in a typical beverage. The above-described natural carbohydrates may be monosaccharides such as glucose and fructose, disaccharides such as maltose and sucrose, polysaccharides such as dextrin and cyclodextrin, and sugar alcohols such as xylitol, sorbitol, and erythritol. As a sweetener, it is possible to use a natural sweetener such as thaumatin and stevia extract, a synthetic sweetener such as saccharin and aspartame, and the like. The proportion of the natural carbohydrates is generally about 0.01 to 0.20 g, or about 0.04 to 0.10 g per 100 ml of the composition of the present invention.

In addition to the aforementioned ingredients, the composition of the present invention may contain various nutrients, vitamins, electrolytes, flavors, colorants, pectic acids and salts thereof, alginic acid and salts thereof, organic acids, protective colloid thickeners, pH adjusters, stabilizers, preservatives, glycerin, alcohols, carbonating agents used in carbonated drinks, and the like. In addition, the composition of the present invention may contain flesh for preparing natural fruit juice, fruit juice drinks, and vegetable drinks. These ingredients may be used either alone or in combinations thereof. The proportion of these additives is not significantly important, but is generally selected within a range of 0.01 to 0.20 part by weight per 100 parts by weight of the composition of the present invention.

In the present disclosure, the composition for colonization of external probiotics in the gut is administered in a pharmaceutically or sitologically effective amount. In the present invention, “the pharmaceutically or sitologically effective amount” refers to an amount sufficient to treat or alleviate diseases or symptom at a reasonable benefit/risk ratio applicable to medical treatment, and an effective dosage level may be determined according to factors including types of diseases of patients, the severity of disease, the activity of drugs, sensitivity to drugs, administration time, administration route, excretion rate, treatment period, and simultaneously used drugs, and factors well known in other medical fields.

The composition for colonization of external probiotics in the gut according to the present disclosure may be administered individually or sequentially or simultaneously in combination with other agents, and multiple may be administered single or multiple doses. It is important to administer the amount that can obtain the maximum effect with the minimum amount without side effects in consideration of all of the above factors, which can easily be determined by those skilled in the art to which the present disclosure pertains.

The composition for colonization of external probiotics in the gut according to the present disclosure may be administered to a subject by various routes. All modes of administration can be expected, for example, the composition may be administered by oral administration, subcutaneous injection, intraperitoneal administration, intravenous administration, intramuscular injection, paraspinal space (intrathecal) injection, sublingual administration, buccal administration, intrarectal administration, intravaginal administration, intraocular administration, otic administration, nasal administration, inhalation, spraying through the mouth or nose, dermal administration, transdermal administration, etc.

The composition for colonization of external probiotics in the gut according to the present disclosure may be determined according to the type of active ingredients together with several related factors such as the disease or condition to be treated or alleviated, the route of administration, the patient's age, sex, weight, and the severity of the disease or symptom.

Another aspect of the present disclosure provides a kit for colonization of external probiotics in the gut, which comprises:

a first container comprising the first composition;

a second container comprising a second composition; and

an instruction manual.

In the present disclosure, the first container and the second container may serve to package each of the first composition and the second composition, and may also serve to store and fix the same. The material of the first container and the second container may be, for example, plastic, aluminum, glass bottle, vinyl, paper, etc., but is not limited thereto.

In the present disclosure, the formulation of the first composition or the second composition may be one or more selected from the group consisting of powders, granules, sustained-release granules, enteric granules, solutions, emulsions, suspensions, spirits, troches, perfumes, tablets, sustained-release tablets, enteric tablets, sublingual tablets, hard capsules, soft capsules, sustained-release capsules, enteric capsules, pills, tinctures, soft extracts, dried extracts, liquid extracts, injections, and perfusion solutions, but is not limited thereto.

In the kit of the present disclosure, the first composition may be administered to a subject to form a functional group in intestinal mucosa by a precursor imparting a bioorthogonal functional group. After a bioorthogonal functional group is formed in the intestinal mucosa by the first composition, through administration of the second composition, probiotics which are modified with a functional group that selectively binds to a bioorthogonal functional group, may bind to intestinal mucosa and enter the intestine through a bioorthogonal click chemistry reaction. Such introduction of external probiotics through bioorthogonal click chemistry into the intestine allows to overcome the discharge of external probiotics and colonization resistance by binding to and colonizing the intestinal membrane through chemical reactions, and allows to stay in the intestine for a long time and improve the intestinal environment.

In the kit of the present disclosure, each of the first composition contained in the first container and the second composition contained in the second container may be used one or more times without limitation. When the kit is first administered, the second composition may be administered after the first composition is administered in advance, and thereafter, each composition may be administered simultaneously or sequentially.

In the present disclosure, the instruction manual may include instructions relating to colonizing external probiotics in the intestine, instructions may be written on a sheet or booklet separate from the first container and the second container, and the sheet or booklet may be included in the first container and the second container together.

In the present disclosure, the instruction manual may describe that the first composition must be first administered to a subject in need thereof, and then, the second composition must be first administered 12 to 48 hours thereafter, 12 to 42 hours thereafter, 12 to 36 hours thereafter, 12 to 30 hours thereafter, 12 to 24 hours thereafter, 18 to 48 hours thereafter, 18 to 42 hours thereafter, 18 to 36 hours thereafter, 18 to 30 hours thereafter, 18 to 24 hours thereafter, 24 to 48 hours thereafter, 24 to 42 hours thereafter, 24 to 36 hours thereafter, 24 to 30 hours thereafter, 24 to 26 hours thereafter, 12 hours thereafter, or 24 hours thereafter, but the instruction is not limited thereto.

The following may be additionally described in the instruction manual: after the first administration of the first composition, the first composition and the second composition may be co-administered, and the second composition may be administered after administration of the first composition in the same way as the first administration; the second composition may be repeatedly administered 1 to 3 times, 2 to 3 times, 1 time, 2 times, or 3 times, in which the second composition may be administered for 1 to 10 days, 1 to 9 days, 1 to 8 days, 1 to 7 days, 2 to 10 days, 2 to 9 days, 2 to 8 days, 2 to 7 days, 3 to 10 days, 3 to 7 days, 4 to 10 days, 4 to 7 days, 5 to 10 days, 5 to 7 days, at intervals of 1 day, 2 days, 3 days, 4 days, 5 days, 6 days, 7 days, 8 days, 9 days, or 10 days, but the administration is not limited thereto. For example, it may be possible that the second composition is administered 24 hours after the first administration of the first composition; the second composition may be administered alone 7 days and 14 days after the first administration of the second composition, respectively; and after 21 days, the first composition and the second composition may be co-administered or the second composition may be administered 24 hours after administration of the first composition. Thereafter, the second composition may be repeatedly administered alone for a single administration of the first composition in the same manner as above, but the method of administration is not limited to the conditions described in the above embodiments.

Still another aspect of the present disclosure provides a method for colonization of external probiotics in the gut, which comprises administering to a subject in need thereof a composition comprising, as active ingredients, a first composition comprising a precursor that imparts a bioorthogonal functional group to the intestinal mucous membrane, and a second composition comprising probiotics modified with a functional group that selectively binds to the bioorthogonal functional group.

Still another aspect of the present disclosure provides a method for colonization of external probiotics in the gut, which comprises administering a first composition comprising a precursor that imparts a bioorthogonal functional group to the intestinal mucous membrane to a subject in need thereof; and administering a second composition comprising probiotics modified with a functional group that selectively binds to the bioorthogonal functional group to the subject.

Still another aspect of the present disclosure provides a method for treating or alleviating inflammatory bowel disease, irritable metabolic syndrome, diabetes, hypertension, autoimmune disease, Alzheimer's disease, or cancer, which comprises administering a first composition comprising a precursor that imparts a bioorthogonal functional group to intestinal mucosa to a subject in need thereof; and administering a second composition comprising probiotics modified with a functional group that selectively binds to the bioorthogonal functional group to the subject.

Still another aspect of the present disclosure provides a use of a composition for colonization of external probiotics in the gut, in which the composition comprises, as active ingredients, a first composition comprising a precursor that imparts a bioorthogonal functional group to the intestinal mucosa; and a second composition comprising probiotics modified with a functional group that selectively binds to the bioorthogonal functional group.

Still another aspect of the present disclosure provides a use of a first composition comprising a precursor that imparts a bioorthogonal functional group to the intestinal mucosa and a second composition comprising probiotics modified with a functional group that selectively binds to the bioorthogonal functional group for the preparation of an agent for colonization of external probiotics in the gut.

Still another aspect of the present disclosure provides a use of a composition for the preparation of an agent for colonization of external probiotics in the gut, in which the composition comprises, as active ingredients, a first composition comprising a precursor that imparts a bioorthogonal functional group to the intestinal mucosa; and a second composition comprising probiotics modified with a functional group that selectively binds to the bioorthogonal functional group.

In the present disclosure, the term “subject” refers to an object that requires the treatment of a disease, and more specifically, mammals such as humans or non-human primates, mice, rats, dogs, cats, horses, and cows.

In the present disclosure, the term “treatment” refers to all actions that improve or beneficially change the disease of a target disease and metabolic disorder symptoms resulting therefrom by the administration of the pharmaceutical composition according to the present disclosure.

In the present disclosure, the term “alleviation” refers to all actions that reduce the parameters associated with a target disease (e.g., degree of symptoms) by the administration of the composition according to the present disclosure.

In the present disclosure, the term “administration” refers to provision of the predetermined composition of the present disclosure to a subject by any appropriate method.

In the present disclosure, when the term “comprising” is used, it means that it may further include other components, instead of excluding other components, unless particularly described otherwise. The term “a step of (˜ing)” or the term “a step of ˜ing” used throughout the entirety of the present disclosure does not mean “a step for ˜”.

Hereinafter, preferred Examples and Experimental Examples are presented to help understanding of the present disclosure. However, the following Examples and Experimental Examples are only provided for easier understanding of the present disclosure, and the contents of the present disclosure are not limited by these Examples and Experimental Examples.

### EXAMPLES

### Example 1

### Introduction and Characterization of DBCO of External Probiotics

In order to prepare probiotics (DBCO-Bac.) into which dibenzocyclooctyne (DBCO) is introduced, 10¹¹ Lactobacillus acidophilus were separated from the culture medium through a centrifuge (4,000×g, 4° C., 15 minutes) and washed once in saline solution at 37° C., and 1 mL of saline solution in which N-hydroxysuccinimide (NHS)-DBCO (DBCO-NHS) is dissolved and 1 mL of a Lactobacillus acidophilus solution were mixed, and reacted in an orbital shaker (80 rpm) at 4° C. for 1 hour. After completion of the reaction, Lactobacillus acidophilus was separated from the solution through a centrifuge (4,000×g, 4° C., 15 minutes), and washed twice in saline solution at 4° C. The DBCO introduced was subjected to fluorescent labeling by culturing at room temperature in saline solution containing 100 μM fluorescein isothiocyanate-linked azide (FITC-N₃) for 30 minutes, washed twice with physiological saline at 4° C., and evaluated through fluorescence imaging and flow cytometry.

### Example 2

### Introduction and Characterization of Azide Group by Metabolic Engineering of Goblet Cell-Like Cells

In order to evaluate the introduction of an azide group (—N₃) (i.e., a functional group for bioorthogonal click chemistry) to the intestinal mucosa, HT29-MTX-e12 cells, which are goblet cell-like cells in vitro, were used. An azide group was introduced to the cell surface through glycan manipulation using tetraacetylated N-azidoacetyl-D-mannosamine (ManNAz) and tetraacetylated N-azidoacetyl-D-galactosamine (GalNAz), which are derivatives of mannosamine and galactosamine, which is a molecule used for mucosal formation. Specifically, HT29-MTX-e12 cells were cultured for 24 hours in DMEM medium containing 100 μM ManNAz or GalNAz. The azide group introduced was subjected to fluorescent labeling by culturing for 30 minutes in a medium containing 10 μM DBCO-Cy5.5, washed twice with physiological saline at 4° C., reacted with 20 μM Hoechst 33342 for 15 minutes, and then the cell nuclei were stained and observed.

### Example 3

### Western Blot Analysis of Azide Group Introduced

For quantitative evaluation of an azide group according to the precursor treatment concentration, HT29-MTX-e12 cells in 10 mL medium without sugar or containing various concentrations of GalNAz or ManNAz were inoculated onto 60 mm×15 mm polystyrene tissue culture plates at a density of 2×10⁶ cells per plate, and cultured for 24 hours. The cells were obtained after washing with PBS (pH 7.4), lysed in a cell lysis solution (50 mM Tris, pH 7.2, 150 mM NaCl, 1% Triton X-100, 200 ng/mL phenylmethylsulfonyl fluoride) containing phosphatase inhibitor cocktail (St. Louis, Mo., USA), and 10 μL of phosphine-PEG-biotin (Waltham, Mass., Thermofisher Scientific) per 100 μL of the lysate was reacted at room temperature for 12 hours. Then, after subjecting the resultant to electrophoresis on 10% SDS polyacrylamide gel to be transferred to Immobilon P membrane (Millipore Corporation, Billerica, Mass., USA), treated with 1,000:1 diluted HRP-streptavidin (Waltham, Mass., Thermofisher Scientific) at room temperature for 2 hours, and confirmed by developing on Pierce™ECL Western blotting substrate (Waltham, Mass., Thermofisher Scientific).

### Example 4

### 3-Dimensional Intestinal Mucosal Simulation Model

To evaluate the binding ability of DBCO-Bac. to the mucosa in a three-dimensional environment, mucosal formation of HT29-MTX-e12 cells was promoted through N-[N-(3,5-difluorophenacetyl)-L-alanyl]-S-phenylglycine t-butyl ester (DAPT) stimulation, and a 3D intestinal mucosal simulation model was prepared using the same. HT29-MTX-e12 cells were inoculated in DMEM medium and cultured for 24 hours, treated with 20 μM DAPT 4 times at intervals of once every 2 days, and from the second DAPT treatment, treated with the culture medium containing 100 μM GalNAz or ManNAz three times at intervals of once every two days. Then, the resultant was treated with DBCO-Bac. at a concentration of 5×10⁸/mL.

### Example 5

### Introduction of Azide Group into Mouse Intestinal Mucosa and Evaluation of Intestinal Mucosal Binding Ability of Probiotics

To confirm the expression level and expression time of an azide group in the intestinal mucosa, GalNAz or ManNAz was orally administered to mice, and the expression level of an azide group in the intestinal mucosa was evaluated through the binding ability with DBCO-Cy5.5. 5 mg of GalNAz or ManNAz was orally administered per animal, the intestinal tissue was divided into 4 parts (duodenum, jejunum, ileum, and colon) of the small intestine and extracted after 12 hours, 24 hours, 36 hours, and 48 hours at intervals of 12 hours, and dipped in 20 μM of each DBCO-Cy5.5 solution and stained.

In addition, in order to evaluate the intestinal mucosal binding ability of DBCO-Bac., GalNAz or ManNAz per animal was orally administered to 5 mg mice, and 24 hours thereafter, DBCO-Bac. was administered at a concentration of 10⁹/mL.

### EXPERIMENTAL EXAMPLES

### Experimental Example 1

### Confirmation of Introduction of DBCO in Probiotics

DBCO, which can react with an azide group in vivo without a catalyst, was introduced to the surface of probiotics by bioorthogonal click chemistry by the method of Example 1 above. DBCO-NHS was conjugated to an amine group on the surface of Lactobacillus acidophilus, and the DBCO introduced was labeled using FITC-N₃.

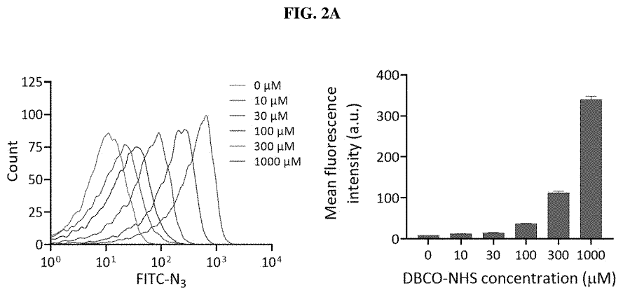

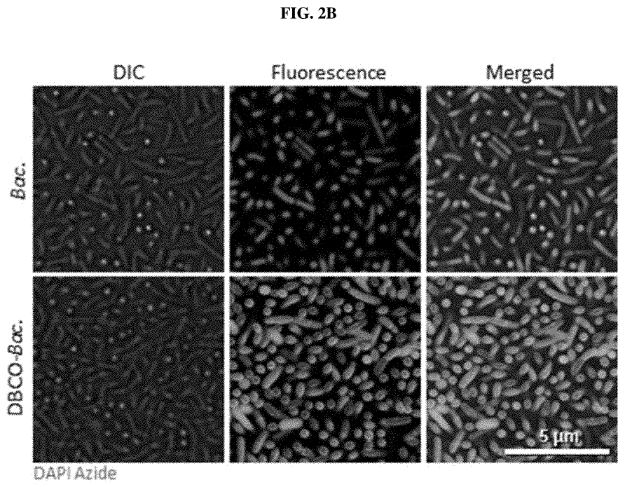

Then, as a result of performing flow cytometry, the fluorescence intensity of the labeled FITC increased as the concentration of DBCO increased as shown in FIG. 2A. This shows that FITC was DBCO-specifically introduced. According to the results of FIG. 2A, the DBCO introduction condition was determined as the DBCO 1,000 μM condition, which showed the highest fluorescence intensity. In addition, as a result of confirming the fluorescence image through the confocal microscope, it was confirmed that green fluorescence appeared on the surface of probiotics (Bac.) as shown in FIG. 2B, and through the same, it was found that the DBCO introduced was selectively distributed on the surface of the probiotics.

### Experimental Example 2

### Confirmation of the Structure and Viability of Probiotics Following Introduction of DBCO

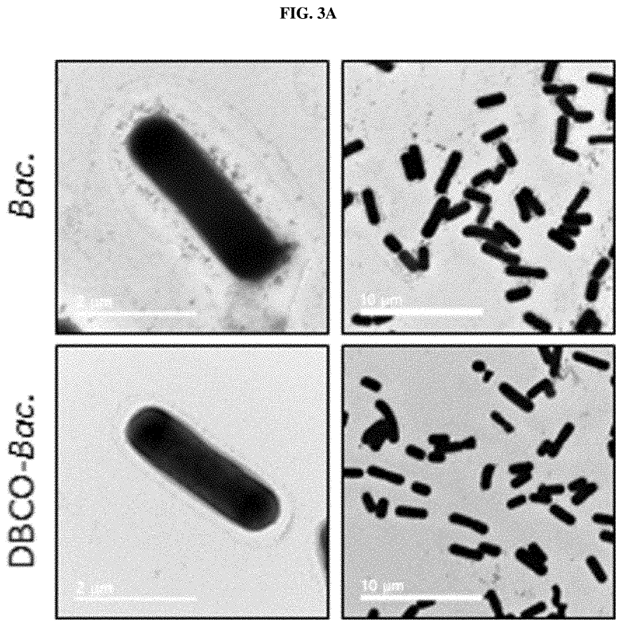

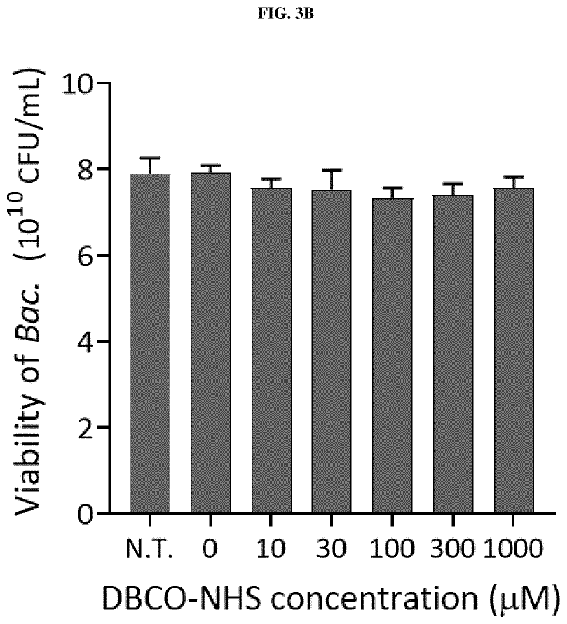

In order to determine whether the introduction of DBCO affects the structural characteristics of probiotics, the probiotics were observed with a transmission electron microscope, and as a result, it was confirmed that the structural malformation of the probiotics did not occur by the introduction of DBCO as shown in FIG. 3A.

Additionally, in order to evaluate the effect of DBCO introduction on the activity of probiotics, the colony forming unit (CFU) measurement method was performed. The probiotics prepared were smeared on MRS agar medium using a serial dilution method, and 48 hours thereafter, the viability was evaluated by counting the number of colonies formed on the plate. As a result of evaluating the viability of probiotics according to the concentration of DBCO (0 μM, 10 μM, 30 μM, 100 μM, 300 μM, and 1,000 μM), there was no significant difference in the viability of probiotics by the introduction of DBCO as shown in FIG. 3B.

These results show that the present disclosure can increase intestinal colonization without functional loss of probiotics.

### Experimental Example 3

### Confirmation of Expression of Azide Group in Goblet Cell-Like Cells

In the case of introduction of an azide group into the intestinal mucosa, after introduction of an azide group into the HT29-MTX-e12 goblet cell-like cell line by the method of Example 2, the binding ability with DBCO-Cy5.5 was evaluated.

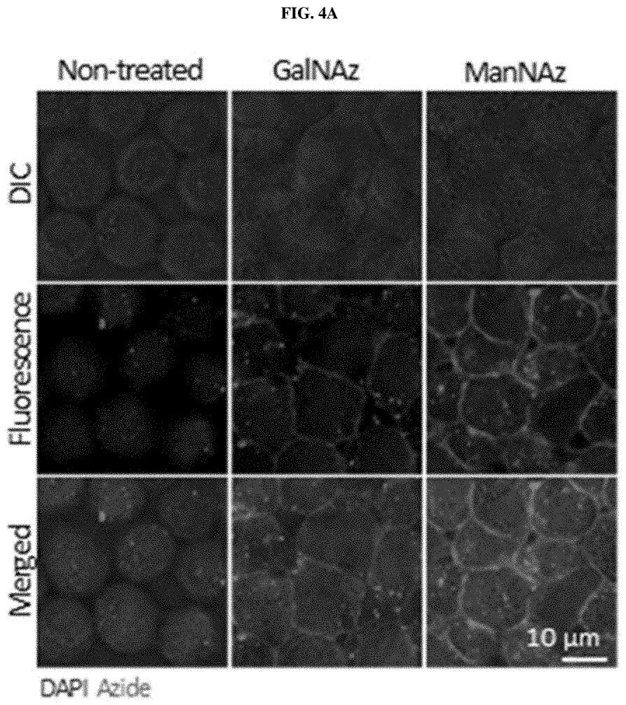

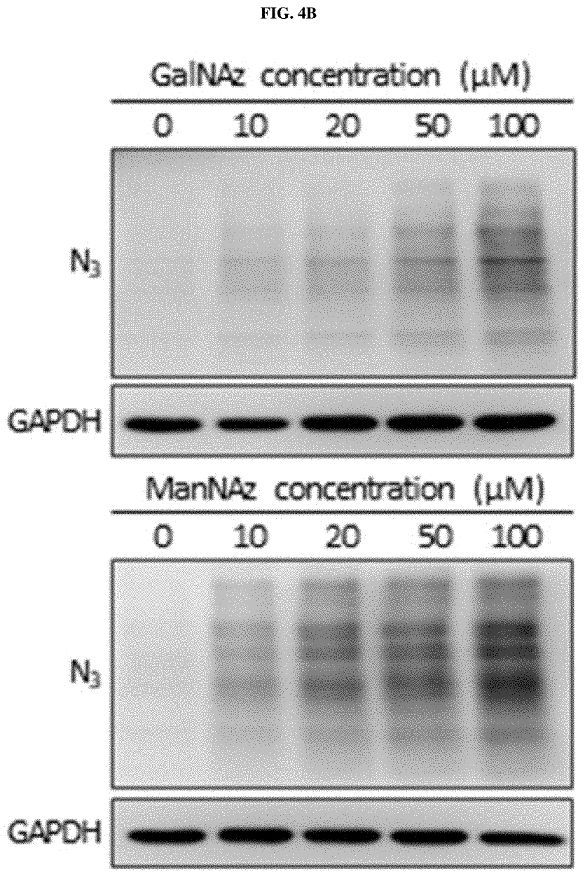

After introducing an azide group into a goblet cell-like cell line, which is a secreting cell of intestinal mucosa, fluorescence images and Western blots were confirmed. As a result, it was confirmed that red fluorescence appeared in HT29-MTX-e12 cells upon treatment with GalNAz or ManNAz as shown in FIG. 4A, and it was confirmed that the generation of an azide group increased depending on the concentration of GalNAz and ManNAz. as shown in FIG. 4B.

From these results, it was confirmed that an azide group, which is a functional group for bioorthogonal click chemistry, was generated in goblet cell-like cells by metabolic glycoengineering.

### Experimental Example 4

### Evaluation of Binding Ability of Azide Group Introduced into Goblet Cell-Like Cells and DBCO-Bac.

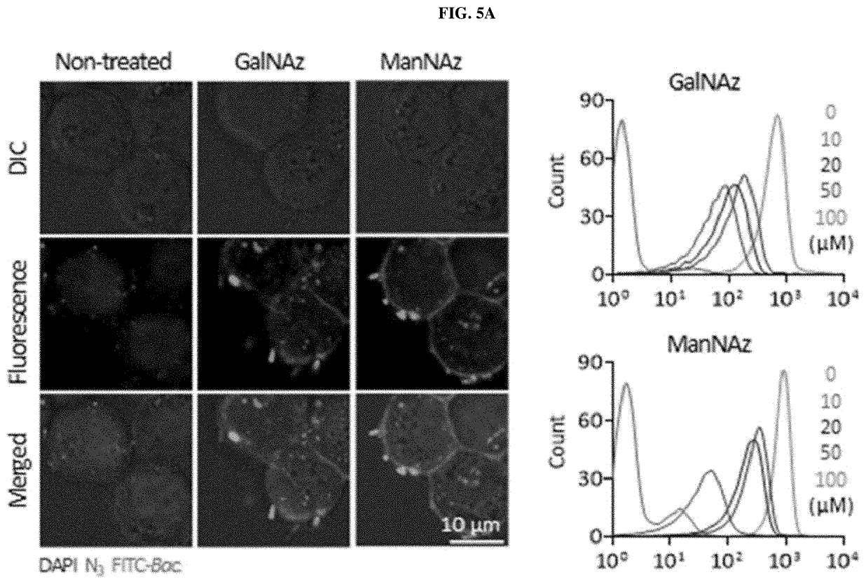

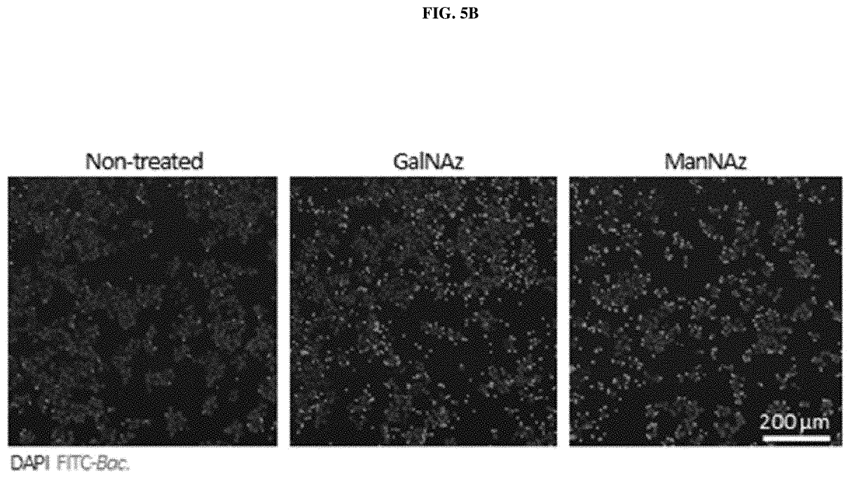

When DBCO-Bac. is treated on goblet cell-like cells into which an azide group has been introduced in Experimental Example 3, red fluorescence representing an azide group and green fluorescence representing DBCO-Bac. appear clearly as shown in FIG. 5A. Therefore, it was confirmed that the binding ability to DBCO-Bac. increased significantly compared to the control group into which an azide group was not introduced, and particularly for cells treated with 100 μM of GalNAz, it was confirmed that the binding ability to DBCO-Bac. increased by about 400 times compared to the control group and by about 500 times compared to the control group when treated with 100 μM of ManNAz.

Additionally, when the low-magnification fluorescence imaging results were confirmed, in the case of HT29-MTX-e12 cells treated with GalNAz or ManNAz, green fluorescence increased compared to the control group as shown in FIG. 5B; therefore, overall, it was confirmed that the distribution of DBCO-Bac. in the culture dish increased.

These results indicate that the present disclosure can allow only the external probiotics treated with DBCO to selectively bind when subjected to metabolic glycoengineering, and this is very meaningful in that it enables selective introduction of specific external probiotics without affecting colonization resistance for inhibition of pathogens in vivo.

### Experimental Example 5

### Evaluation of Binding Ability of DBCO-Bac. to Intestinal Mucosa in 3-Dimensional Intestinal Mucosal Simulation Model

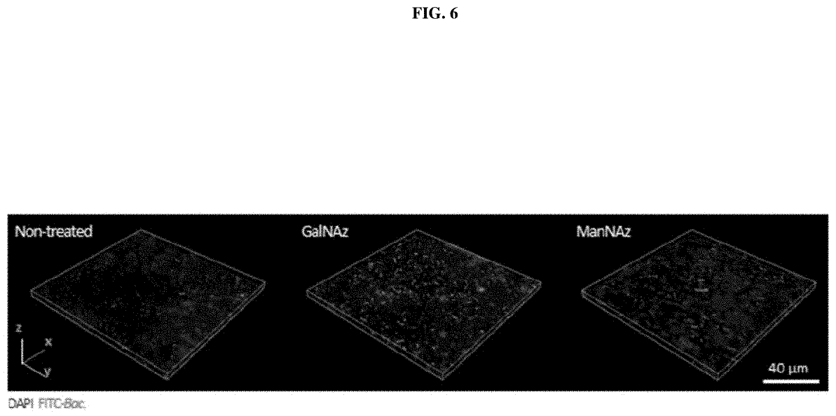

Additionally, in order to evaluate the binding ability of DBCO-Bac. to the intestinal mucosa in a three-dimensional environment, a three-dimensional intestinal mucosal simulation model was prepared using HT29-MTX-e12 cells in which mucosal formation was promoted through DAPT stimulation by the method of Example 4 above. When the 3D fluorescence image result was confirmed, in the case of treatment with 100 μM GalNAz or ManNAz, green fluorescence increased significantly compared to the control group as shown in FIG. 6; therefore, it was confirmed that the binding ability to DBCO-Bac. was improved. In particular, it was confirmed that binding ability increased not only to the surface but also to the interior of the mucosal structure; therefore, it was found that the present disclosure can effectively introduce specific external probiotics even in structures such as the intestinal mucosa composed of three dimensions.

### Experimental Example 6

### Confirmation of Expression of Azide Group in Mouse Intestinal Mucosa

In the case of introducing an azide group into the mouse intestinal mucosa, after oral administration of GalNAz or ManNAz to mice by the method of Example 5 above, the expression level of an azide group in the intestinal mucosa was evaluated through the binding ability with DBCO-Cy5.5.

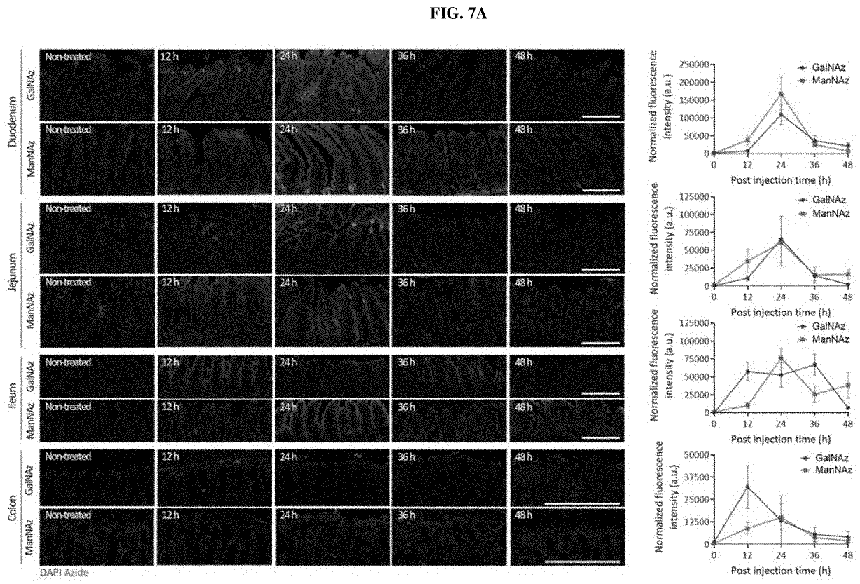

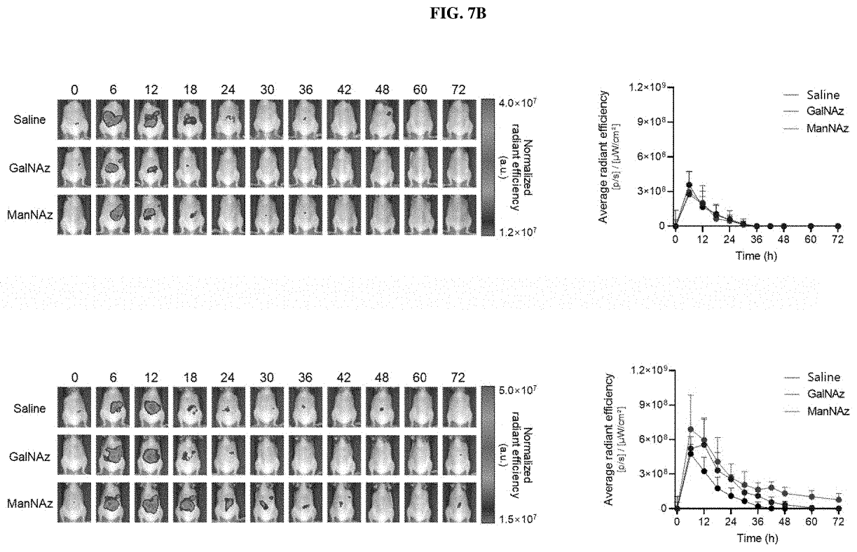

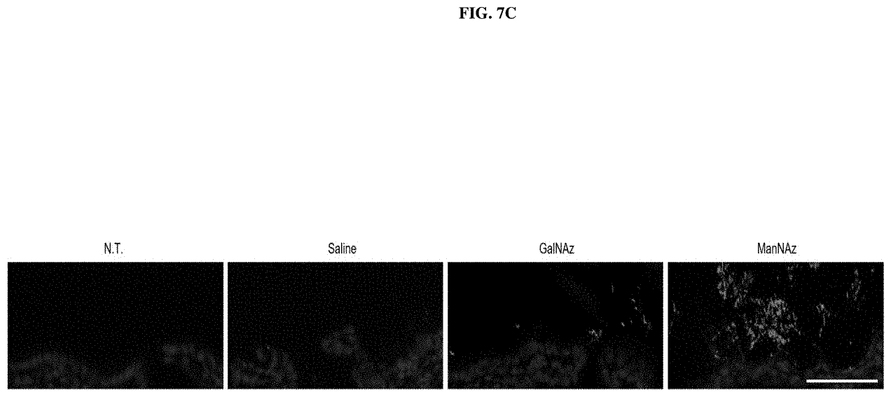

As a result of confirming the expression level of an azide group over time in the duodenum, jejunum, ileum, and colon of the small intestine after oral administration of GalNAz or ManNAz, it was confirmed that most of the fluorescence intensity was strongest when about 24 hours elapsed after oral administration of GalNAz or ManNAz in each region of the intestine as shown in FIG. 7A. That is, it was found that the expression level of an azide group was high in the intestinal mucosa when about 24 hours elapsed after administration of GalNAz or ManNAz.

### Experimental Example 7

### Confirmation of Binding Ability of Azide Group Introduced into Mouse Intestinal Mucosa and DBCO-Bac.

GalNAz and ManNAz was orally administered to mice by the method of Example 5, respectively, and then Bac. without DBCO modification and DBCO- modified Bac. (DBCO-Bac.) were treated, respectively, and the fluorescence values of Bac. remaining in the intestinal mucosa over time were compared.

As a result, it was confirmed that when treated with Bac. without DBCO modification, the amount of fluorescence of Bac. remaining in the intestinal mucosa almost disappeared in both GalNAz and ManNAz administration groups after about 24 hours similar to the saline administration group used as a control group as shown in FIG. 7B (the drawing on top of FIG. 7B).

In contrast, it was confirmed that when treated with DBCO-Bac., the amount of fluorescence remaining in the intestinal mucosa was high and the fluorescence maintained for a long time in both the GalNAz and ManNAz administration groups, compared to the physiological saline-treated group (the drawing on bottom of FIG. 7B).

From these results, it was found that when DBCO-modified Bac. was administered to mice, DBCO could bind to azide groups introduced into the intestinal mucosa by GalNAz and ManNAz, and retain a large amount of Bac. in the intestinal mucosa for a long time.

Experimental Example 8. Confirmation of binding ability of DBCO-Bac. with azide group introduced into intestinal mucosa when several bacterial groups are administered simultaneously

In order to confirm whether bioorthogonal click chemistry of DBCO and an azide group is applicable when several groups of bacteria were administered at once as in probiotics, GalNAz and ManNAz were each orally administered to mice by the method of Example 5, and simultaneously treated with Bifidobacterium longum modified with DBCO and Lactobacillus acidophilus modified with DBCO, and the fluorescence value of Bac. remaining in the intestinal mucosa was confirmed.

As a result, it was confirmed that, compared to the control group, the amounts of Bifidobacterium longum (orange fluorescence) and Lactobacillus acidophilus (green fluorescence) remaining in the intestinal mucosa were higher in the GalNAz and ManNAz administration groups as shown in FIG. 7C, and particularly, it was confirmed that the amount of bacteria remaining in the intestinal mucosa was significantly higher in the ManNAz-administered group.

From these results, it was found that even when several groups of bacteria are administered at once, DBCO binds to an azide group introduced into the intestinal mucosa regardless of their type, and thereby external probiotics modified with DBCO can colonize the intestinal mucosa.

The description of the present disclosure above is for illustration purpose only, those skilled in the art to which the present disclosure belongs will be able to understand that it can easily be modified into other specific forms without changing the technical spirit or essential characteristics of the present disclosure. Therefore, it should be understood that the Examples described above are illustrative in all respects and not limitative.

1. A composition for colonization of external probiotics in the gut, which comprises as active ingredients:
a first composition comprising a precursor that imparts a bioorthogonal functional group to the intestinal mucosa; and
a second composition comprising probiotics modified with a functional group that selectively binds to the bioorthogonal functional group.

2. The composition of claim 1, wherein the first composition, when administered to a subject, forms a functional group on the intestinal mucosa by a precursor that imparts a bioorthogonal functional group to thereby change the composition of the intestinal mucosa, and
the second composition, when administered to a subject, enables selective colonization in the intestinal mucosa of the probiotics modified with a functional group that selectively binds to a bioorthogonal functional group through a specific chemical reaction.

3. The composition of claim 2, wherein the specific chemical reaction is a bioorthogonal click chemistry reaction.

4. The composition of claim 1, wherein the bioorthogonal functional group is an azide group (—N₃).

5. The composition of claim 1, wherein the precursor that imparts the bioorthogonal functional group is one or more selected from the group consisting of tetraacetylated N-azidoacetyl-D-galactosamine (GalNAz), tetraacetylated N-azidoacetyl-D-mannosamine (ManNAz), tetraacetylated N-azidoacetyl-D-glucosamine, tetraacetylated N-azidoacetyl-D-galactose, tetraacetylated N-azidoacetyl-D-mannose, and tetraacetylated N-azidoacetyl-D-glucose.

6. The composition of claim 1, wherein the functional group that selectively binds to a bioorthogonal functional group is one or more selected from the group consisting of dibenzocyclooctyne (DBCO), cyclooctyne, 2,2-difluoro-1,3-cyclooctanedione, difluorinated cyclooctyne, bicyclo[6.1.0]non-2-yne or a derivative thereof, 4-dibenzocyclooctinol,

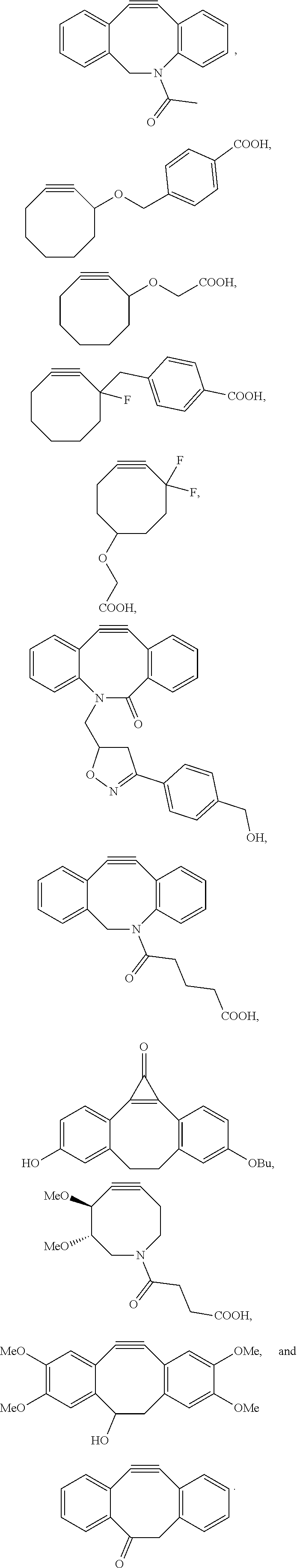

7. The composition of claim 1, wherein the composition introduces external probiotics into the intestine and improves the intestinal environment while maintaining the structure and function of the external probiotics.

8. The composition of claim 1, wherein the molar concentration of the functional group that selectively binds to the bioorthogonal functional group of the second composition is 3-12 fold that of the precursor imparting the bioorthogonal functional group of the first composition relative to the composition.

9. A kit for colonization of external probiotics in the gut, which comprises:
a first container comprising the first composition of claim 1;
a second container comprising a second composition; and
an instruction manual.

10. The kit of claim 9, wherein the formulation of the first composition or second composition is one or more selected from the group consisting of powders, granules, sustained-release granules, enteric-coated granules, solutions, emulsions, suspensions, spirits, troches, perfumes, tablets, sustained-release tablets, enteric-coated tablets, sublingual tablets, hard capsules, soft capsules, sustained-release capsules, enteric- coated capsules, pills, tinctures, soft extracts, dried extracts, fluid extracts, injections, and perfusion solutions.

11. The kit of claim 9, wherein the instruction manual describes that the first composition is first administered to a subject in need thereof, and 12 to 48 hours thereafter, the second composition is first administered.

12. The kit of claim 11, wherein the instruction manual further describes that after the first administration of the first composition, it is possible to administer the first composition and the second composition in combination or to administer the second composition after the administration of the first composition.

13. The kit of claim 11, wherein the instruction manual further describes that it is possible to administer the second composition 2 to 3 times repeatedly per one administration of the first composition.

14. A method for colonization of external probiotics in the gut, which comprises:
administering a first composition comprising a precursor that imparts a bioorthogonal functional group to the intestinal mucosa to a subject in need thereof; and
administering a second composition comprising probiotics modified with a functional group that selectively binds to the bioorthogonal functional group to the subject.
